(19) United States
(12) Patent Application Publication
(10) Pub. No.: US 2026/0114848 A1
(43) Pub. Date: Apr. 30, 2026

(54) METHOD FOR PROVIDING INFORMATION ABOUT DOPPLER ULTRASOUND IMAGE, AND DEVICE FOR PROVIDING INFORMATION ABOUT DOPPLER ULTRASOUND IMAGE USING SAME

(71) Applicant: ONTACT HEALTH CO., LTD., Seoul (KR)

(72) Inventors: Jae Ik JEON, Uijeongbu-si Gyeonggi-do (KR); Hack Joon SHIM, Seoul (KR); Ji Yeon KIM, Seoul (KR); Seong Min HA, Seoul (KR); Annes CHOI, Seoul (KR)

(73) Assignee: ONTACT HEALTH CO., LTD., Seoul (KR)

(21) Appl. No.: 19/100,129

(22) PCT Filed: Aug. 18, 2023
(86) PCT No.: PCT/IB2023/058265

§ 371 Date: Nov. 20, 2025

(30) Foreign Application Priority Data
Aug. 1, 2022 (KR) 10-2022-0095284

(51) Int. Cl. A61B 8/00; A61B 8/06; A61B 8/08; G06V 10/40; G06V 10/764; G06V 10/82

(52) U.S. Cl. CPC A61B 8/463; A61B 8/065; A61B 8/0883; A61B 8/488; A61B 8/5207; G06V 10/40; G06V 10/764; G06V 10/82; G06V 2201/031

(57) ABSTRACT

Abstract as amended below shows added text with underlining and deleted text with The present disclosure provides a method, implemented by a processor, for providing information about a doppler ultrasound image, and a device using the same, the method including receiving a first image showing a portion of an individual's heart and a second image showing the flow or velocity of blood; and classifying a view of the second image on the basis of the received first and second images by using a view classification model trained to classify a view by using each of the first and second images as inputs.

### TECHNICAL FIELD

The present disclosure relates to a method for providing information about a doppler ultrasound image and a device for providing information about a doppler ultrasound image using the same.

### BACKGROUND ART

A cardiac ultrasound examination is performed by projecting ultrasound waves on a three-dimensional structure of a heart in multiple planes to acquire images of the heart and measure hemodynamic variables.

In this case, a medical staff positions an ultrasound probe in a position where it is easy to acquire doppler ultrasound images to acquire multi-faceted images through the anatomical structures around the heart, such as between ribs, and records the images by finding an appropriate slice through rotation and tilting.

Meanwhile, among cardiac ultrasound modes, in a doppler mode, it is possible to measure the velocity of blood flow in blood vessels. In particular, a blood flow measurement method based on the doppler mode of ultrasound has the characteristic of being able to measure blood flow velocity in real time noninvasively, and is widely used in modern medical diagnosis.

In this case, the doppler ultrasound image may provide blood flow information for various views. Typically, doppler images acquired during cardiac ultrasound only provide velocity-time information for blood vessels.

That is, during an image analysis stage, the medical staff should classify which view the acquired doppler ultrasound image corresponds to.

Here, since the deviation in the classification of views of doppler ultrasound images acquired depending on the skill level of the medical staff may be large, there is a continuous demand for the development of a new information provision system capable of recognizing and classifying views.

The background technology of the present disclosure has been written to facilitate understanding of the present disclosure. It should not be understood that the matters described in the background technology of the present disclosure are recognized as prior art.

### DETAILED DESCRIPTION OF THE INVENTION

### Technical Problem

Meanwhile, in order to solve the above-mentioned problem, the inventors of the present disclosure attempted to develop an information providing system based on an artificial neural network that is trained to recognize views of doppler cardiac ultrasound images and to distinguish between each view.

In particular, the inventors of the present disclosure have been able to recognize and classify views of doppler ultrasound images by applying an artificial neural network.

More specifically, the inventors of the present disclosure have focused on an artificial neural network trained to classify each corresponding view by inputting a doppler echocardiographic image.

Meanwhile, the inventors of the present disclosure have paid more attention to B-mode images, which provide information related to views, for doppler cardiac ultrasound images.

More specifically, the inventors of the present disclosure have attempted to construct an artificial neural network that extracts features for each of a doppler ultrasound image and a B-mode image, and integrates the extracted features to classify a view for a doppler ultrasound image.

As a result, the inventors of the present disclosure have been able to recognize that highly accurate classification of the view is possible without recording the time of examination for the acquired doppler cardiac ultrasound images.

Furthermore, the inventors of the present disclosure have paid more attention to a digital imaging and communications in medicine (DICOM) format having various tagging information in selecting the doppler ultrasound images and B-mode images.

More specifically, the inventors of the present disclosure have noted that various metadata regarding the ultrasound mode of each image can be utilized within an ultrasound image in DICOM format.

As a result, the inventors of the present disclosure have been able to recognize that rapid acquisition of the doppler ultrasound image and B-mode image is possible based on the metadata.

Furthermore, the inventors of the present disclosure have sought to further apply an algorithm for classifying a view based on a doppler flow direction for the doppler ultrasound image, that is, the position of the baseline.

As a result, the inventors of the present disclosure have developed an information providing system for doppler ultrasound images capable of classifying the views of the doppler cardiac ultrasound images based on an artificial neural network.

The inventors of the present disclosure have been able to realize that by providing a new information provision system, unnecessary diagnosis time can be reduced and acquisition of standardized views is possible with an artificial neural network-based system.

That is, the inventors of the present disclosure have expected that by providing a new information provision system, it would be possible to acquire doppler ultrasound images regardless of the skill level of medical staff, and thus provide highly reliable analysis results for the doppler Cardiac ultrasound images.

Accordingly, an object of the present disclosure is to provide a method for providing information about a doppler ultrasound image configured to recognize and classify a view from a received doppler cardiac ultrasound image and B-mode image using an artificial neural network-based classification model, and a device using the same.

The tasks of the present disclosure are not limited to the tasks mentioned above, and other tasks not mentioned will be clearly understood by those skilled in the art from the description below.

### Technical Solution

In order to solve the above-described problem, a method for providing information about a doppler ultrasound image according to one embodiment of the present disclosure is provided. The method for providing information is a method for providing information about a doppler cardiac ultrasound image implemented by a processor, and includes: receiving a first image showing a portion of an individual's heart and a second image showing a flow or velocity of blood; and classifying a view of the second image based on the received first and second images by using a view classification model trained to classify a view using each of the first and second images as input.

According to a feature of the present disclosure, the classification model may include a first feature extraction unit that extracts features using the first image as input, a second feature extraction unit that extracts features using the second image as input, and a full connection unit that integrates features from each of the first feature extraction unit and the second feature extraction unit.

According to another feature of the present disclosure, the view may be a plurality of views, the view classification model may have an output node corresponding to the number of each of the plurality of views, and a probability corresponding to each of the plurality of views for the second image may be output through the output node.

According to still another feature of the present disclosure, the classifying of the view may include acquiring a probability corresponding to each of the plurality of views output through the output node, and determining the view having the highest value among the probabilities corresponding to each of the plurality of views as the view for the second image.

According to still another feature of the present disclosure, the view may include a view for a pulsed wave (PW) doppler mode and a view for a continuous wave (CW) doppler mode. In this case, the classifying of the view may include classifying the view according to the doppler mode of the view for the pulsed wave doppler mode or the view for the continuous wave doppler mode, based on the received first image and the second image, using the view classification model.

According to still another feature of the present disclosure, the second image may include a baseline that serves as a reference for a doppler flow direction, and the method for providing information may further include classifying the view according to the doppler mode in detail based on a position of the baseline after the classifying of the view according to the doppler mode.

According to still another feature of the present disclosure, the view may be a view for the pulsed wave doppler mode, and the classifying in detail may further include determining the view as MV-PW (Apical) when the baseline is positioned at a top of the second image, or determining the view as PV-PW (PSAX, Aortic) or LVOT-PW (A5C/A3C) when the baseline is positioned at a bottom of the second image.

According to still another feature of the present disclosure, the view may be a view for the continuous wave doppler mode, and the classifying in detail may further include determining the view as at least one of MV (MR)-CW (Apical), AV (AS)-CW (Apical), PV (PS)-CW (PSAX/Aortic), and TV (TR)-CW (PLAX/PSAX/Apical) when the baseline is positioned at a top of the second image, or determining the view as at least one of MV (MS)-CW (Apical), AV (AR)-CW (Apical), and PV (PR)-CW (PSAX/Aortic) when the baseline is positioned at a bottom of the second image.

According to still another feature of the present disclosure, the method for providing information may further include: before the receiving, receiving an ultrasound image in a digital imaging and communications in medicine (DICOM) format that displays metadata including tagging information for the second image; and acquiring the first image and the second image based on the metadata.

According to still another feature of the present disclosure, the classifying of the view may include, by using the view classification model, determining a doppler mode view of at least one of pulmonary vein, MV (MS)-CW (Apical), MV (MR)-CW (Apical), MV-PW (Apical), AV (AS)-CW (Apical), AV (AR)-CW (Apical), TV (TS)-CW (PLAX/PSAX/Apical), TV (TR) CW (PLAX/PSAX/Apical), PV (PS)-CW (PSAX/Aortic), PV (PR)-CW (PSAX/Aortic), PV-PW (PSAX, Aortic), LVOT-PW (A5C/A3C), LVOT-CW (A5C/A3C), Septal Annulus PW TDI (A4C), and Lateral Annulus PW TDI (A4C), for the cardiac ultrasound image.

In order to solve the problem as described above, a device for providing information about a doppler ultrasound image according to another embodiment of the present disclosure is provided. The device for providing information includes: a communication unit configured to receive a first image showing a portion of an individual's heart and a second image showing a flow or velocity of blood; and a processor functionally connected to the communication unit. In this case, the processor is configured to classify a view of the second image based on the received first and second images by using a view classification model trained to classify a view using each of the first and second images as input.

In order to solve the problem as described above, a device for providing information about a doppler ultrasound image according to another embodiment of the present disclosure is provided. The device for providing information includes: an ultrasound probe configured to provide a first image showing a portion of an individual's heart and a second image showing a flow or velocity of blood; and a processor functionally connected to the communication unit. In this case, the processor is configured to classify a view of the second image based on the received first image and the second image using a view classification model trained to classify a view using each of the first image and the second image as input.

According to a feature of the present disclosure, the classification model may include a first feature extraction unit that extracts features using the first image as input, a second feature extraction unit that extracts features using the second image as input, and a full connection unit that integrates features from each of the first feature extraction unit and the second feature extraction unit.

According to another feature of the present disclosure, the view may be a plurality of views, and the view classification model may have an output node corresponding to the number of each of the plurality of views. Furthermore, a probability corresponding to each of the plurality of views for the second image may be output through the output node.

According to still another feature of the present disclosure, the processor may be configured to acquire a probability corresponding to each of the plurality of views output through the output node, and determine the view having the highest value among the probabilities corresponding to each of the plurality of cross-section views as the view for the second image.

According to still another feature of the present disclosure, the view may include a view for a pulsed wave (PW) doppler mode and a view for a continuous wave (CW) doppler mode. In this case, the processor may be further configured to classify the view according to the doppler mode of the view for the pulsed wave doppler mode or the view for the continuous wave doppler mode, based on the received first image and the second image, using the view classification model.

According to still another feature of the present disclosure, the second image may include a baseline that serves as a reference for a doppler flow direction. In this case, the processor may be further configured to classify the view according to the doppler mode in detail based on a position of the baseline.

According to another feature of the present disclosure, the view may be a view for the pulsed wave doppler mode, and the processor may be further configured to determine the view as MV-PW (Apical) when the baseline is positioned at a top of the second image, or determine the view as PV-PW (PSAX, Aortic) or LVOT-PW (A5C/A3C) when the baseline is positioned at a bottom of the second image.

According to still another feature of the present disclosure, the view may be a view for the continuous wave doppler mode, and the processor may be further configured to determine the view as at least one of MV (MR)-CW (Apical), AV (AS)-CW (Apical), PV (PS)-CW (PSAX/Aortic), and TV (TR)-CW (PLAX/PSAX/Apical) when the baseline is positioned at a top of the second image, or determine the view as at least one of MV (MS)-CW (Apical), AV (AR)-CW (Apical), and PV (PR)-CW (PSAX/Aortic) when the baseline is positioned at a bottom of the second image.

According to still another feature of the present disclosure, the communication unit may be further configured to receive an ultrasound image in a digital imaging and communications in medicine (DICOM) format that displays metadata including tagging information for the second image, and the processor may be further configured to acquire the first image and the second image based on the metadata.

Specific details of other embodiments are included in the detailed description and drawings.

### Effects of the Invention

The present disclosure provides an information providing system for a doppler ultrasound image based on an artificial neural network configured to classify an ultrasound view using a doppler cardiac ultrasound image and a B-mode image, thereby providing a highly reliable cardiac ultrasound diagnosis result.

More specifically, according to the present disclosure, it is possible to recognize and classify a view by using the B-mode image matched with the doppler ultrasound image together, thereby classifying with high accuracy which view a corresponding doppler image corresponds to.

In particular, according to the present disclosure, it is possible to provide a post-processing algorithm configured to classify the view by considering a doppler flow direction, that is, a position of a baseline, with respect to a result output from an artificial neural network, thereby enabling highly accurate classification of views of various classes of doppler modes.

That is, the present disclosure provides an information providing system for doppler ultrasound images based on an artificial neural network, thereby enabling medical staff to acquire the doppler cardiac ultrasound images and classify the views regardless of their skill level, thereby contributing to more accurate decision-making and treatment planning at the image analysis stage.

The effects according to the present disclosure are not limited to those exemplified above, and further diverse effects are included in the present specification.

### BRIEF DESCRIPTION OF DRAWINGS

FIG. 1 illustrates a system for providing information about a doppler ultrasound image using a device for providing information about a doppler ultrasound image according to one embodiment of the present disclosure.

FIG. 2A is a block diagram illustrating a configuration of a medical staff device according to one embodiment of the present disclosure.

FIG. 2B is a block diagram illustrating a configuration of an information providing server according to one embodiment of the present disclosure.

FIG. 3 illustrates a procedure of a method for providing information about a doppler ultrasound image according to one embodiment of the present disclosure.

FIGS. 4 to 8 exemplarily illustrate the procedure of the method for providing information about a doppler ultrasound image according to one embodiment of the present disclosure.

FIG. 9 is an exemplary diagram illustrating a structure of a view classification model used in the method for providing information according to various embodiments of the present disclosure.

FIGS. 10A to 10C illustrate evaluation results of a view classification model used in a method for providing information according to various embodiments.

### MODES OF THE INVENTION

The advantages of the disclosure and the method for achieving them will become apparent by referring to the embodiments described in detail below together with the accompanying drawings. However, the present disclosure is not limited to the embodiments disclosed below, but may be implemented in various different forms, and these embodiments are provided only to make the disclosure of the present disclosure complete and to fully inform those skilled in the art of the scope of the disclosure.

The shapes, sizes, ratios, angles, numbers, or the like disclosed in the drawings for explaining embodiments of the present disclosure are exemplary, and therefore the present disclosure is not limited to the matters illustrated. In addition, in describing the present disclosure, when it is determined that a detailed description of a related known technology may unnecessarily obscure the gist of the present disclosure, the detailed description will be omitted. When the terms “includes,” “has,” “consists of,” or the like are used in the present specification, other parts may be added unless “only” is used. When a component is expressed in the singular, it includes a case where the plural is included unless there is a specifically explicit description.

When interpreting components, it is interpreted as including the error range even if there is no separate explicit description.

The individual features of the various embodiments of the present disclosure may be partially or wholly coupled or combined with each other, and as can be fully understood by those skilled in the art, various technical connections and operations are possible, and each embodiment may be implemented independently of each other or may be implemented together in a related relationship.

For clarity in the interpretation of the present specification, the terms used in the present specification are defined below.

The term “individual” as used in the present specification may mean any subject from which information about a doppler ultrasound image is provided. Meanwhile, the individual disclosed in the present specification may be any mammal other than a human, but is not limited thereto.

As used herein, the term “ultrasound image” may be a cardiac ultrasound image that can be acquired by a noninvasive method.

Meanwhile, the ultrasound image may be a still cut image or a video composed of a plurality of cuts. For example, the video may be classified into views for each doppler cardiac ultrasound image for each frame of the video according to the method for providing information about a doppler ultrasound image according to one embodiment of the present disclosure. As a result, the present disclosure may provide a streaming service by performing view classification simultaneously with the reception of a doppler cardiac ultrasound image from an image diagnosis device, and may also provide doppler ultrasound image information in real time.

Meanwhile, the ultrasound image may be two-dimensional videos, but are not limited thereto and may also be three-dimensional images.

As used herein, the terms “first image”, “B-mode cardiac ultrasound image”, and “B-mode ultrasound image” may mean an image recorded in a cross-sectional plane by slightly moving a probe in the same plane and radiating the image.

In this case, a B-mode cardiac ultrasound image that matches the doppler ultrasound image may provide information related to the view.

As used herein, the term “second image”, “doppler cardiac ultrasound image” or “doppler ultrasound image” may be an image that can analyze the velocity of blood flow in a blood vessel in a non-invasive manner among various ultrasound modes. In this case, the doppler ultrasound image may provide blood flow information for various views.

As used herein, the “view” refers to a view for the doppler ultrasound image, which may include a view for a pulsed wave (PW) doppler mode and a view for a continuous wave (CW) doppler mode. However, the view is not limited thereto, and may further include a view for a tissue doppler imaging (TDI) mode.

More specifically, the “view for the doppler ultrasound image” may include views of MV-PW (Apical), LVOT-PW (A5C/A3C), PV-PW (PSAX, Aortic) and pulmonary vein for the pulsed wave doppler mode, and may include views of AV (AR)-CW (Apical), PV (PR)-CW (PSAX/Aortic), MV (MS)-CW (Apical), TV (TS)-CW (PLAX/PSAX/Apical), AV (AS)-CW (Apical), PV (PS)-CW (PSAX/Aortic), MV (MR)-CW (Apical), TV (TR)-CW (PLAX/PSAX/Apical), and LVOT-CW (A5C/A3C) for the continuous wave doppler mode. Furthermore, the “view for doppler ultrasound image” may include views of Septal Annulus PW TDI (A4C) and Lateral Annulus PW TDI (A4C) for the tissue doppler imaging mode.

According to a feature of the present disclosure, the ultrasound image may be an image in DICOM format including metadata for the ultrasound image.

Here, the “metadata” may correspond to image DICOM tag information.

For example, in the case of the B-mode ultrasound image and doppler ultrasound image, only the relevant region may be acquired by cropping based on the coordinate values of the DICOM header.

In this case, the metadata may further include information such as whether the doppler ultrasound image is a still image or a video, a color image or a black and white image.

Meanwhile, doppler cardiac ultrasound images for a plurality of views may have ultrasound views recognized and classified within the image regardless of the doppler ultrasound mode by the view classification model.

As used herein, the term “view classification model” may be a model configured to take as input the doppler cardiac ultrasound image and the B-mode ultrasound image matched thereto, and output a view for the doppler ultrasound image.

According to a feature of the present disclosure, the view classification model may be a model trained to classify views in various doppler modes based on a learning doppler cardiac ultrasound image and B-mode image. In this case, the learning doppler cardiac ultrasound image may be an image in which the views in PW, CW, and TDI doppler mode are each labeled.

According to another feature of the present disclosure, the view classification model may be a model having 15 output nodes trained to classify 15 views of pulmonary vein, MV (MS) CW (Apical), MV (MR)-CW (Apical), MV-PW (Apical), AV (AS)-CW (Apical), AV (AR) CW (Apical), TV (TS)-CW (PLAX/PSAX/Apical), TV (TR)-CW (PLAX/PSAX/Apical), PV (PS)-CW (PSAX/Aortic), PV (PR)-CW (PSAX/Aortic), PV-PW (PSAX, Aortic), LVOT-PW (A5C/A3C), LVOT-CW (A5C/A3C), Septal Annulus PW TDI (A4C), and Lateral Annulus PW TDI (A4C) for the doppler ultrasound image. However, the view classification model is not limited thereto.

That is, in various embodiments of the present disclosure, the view classification model may classify the view with high accuracy regardless of the mode of the input doppler ultrasound image.

Meanwhile, the view classification model may be a model based on DBNet, but is not limited thereto. For example, the classification models may be based on at least one algorithm selected from among U-net, VGG net, DenseNet, Fully Convolutional Network (FCN) with encoder-decoder structure, SegNet, DeconvNet, deep neural network (DNN) such as DeepLAB V3+, SqueezeNet, Alexnet, ResNet18, MobileNet-v2, GoogleNet, Resnet-v2, Resnet50, RetinaNet, Resnet101, Inception-v3. Furthermore, the view classification model may be an ensemble model based on at least two algorithm models among the above-mentioned algorithms.

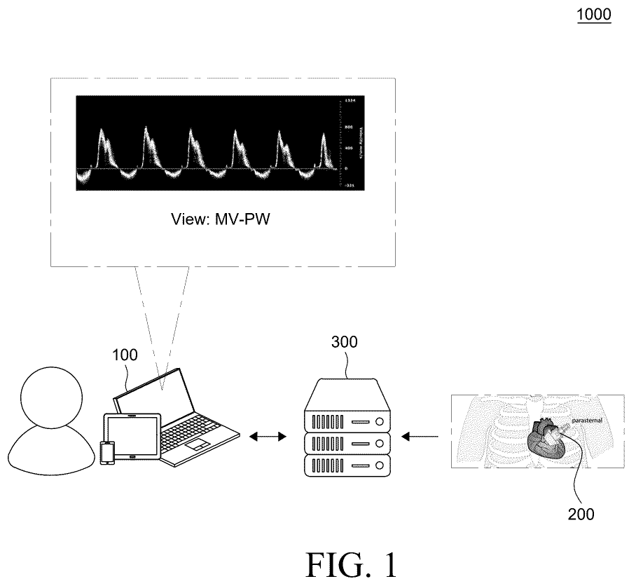

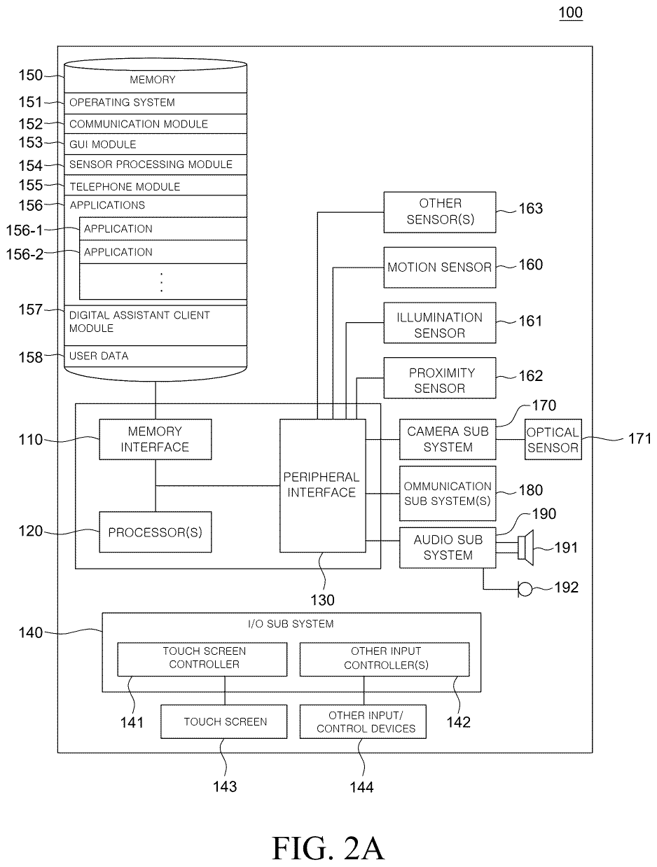

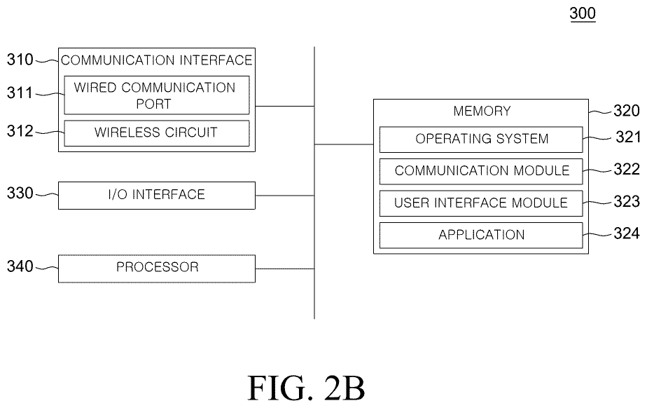

Hereinafter, with reference to FIGS. 1, 2A and 2B, an information providing system for a doppler ultrasound image and a device for providing information about a doppler ultrasound image using a device for providing information about a doppler ultrasound image according to one embodiment of the present disclosure will be described.

FIG. 1 illustrates a system for providing information about a doppler ultrasound image using a device for providing information about a doppler ultrasound image according to one embodiment of the present disclosure. FIG. 2A illustrates an exemplary configuration of a medical staff device for receiving information about the doppler ultrasound image according to one embodiment of the present disclosure. FIG. 2B illustrates an exemplary configuration of the device for providing information about the doppler ultrasound image according to one embodiment of the present disclosure.

First, referring to FIG. 1, an information providing system 1000 may be a system configured to provide information related to a doppler ultrasound image based on a doppler cardiac ultrasound image of an individual. In this case, the information providing system 1000 may include a medical staff device 100 that receives information related to the doppler ultrasound image, a doppler cardiac ultrasound imaging diagnostic device 200 that provides a doppler cardiac ultrasound image, and an information providing server 300 that generates information about the doppler ultrasound image based on the received doppler cardiac ultrasound image.

First, next, the medical staff device 100 may be an electronic device that provides a user interface for displaying information related to the doppler ultrasound image and may include at least one of a smartphone, a tablet PC (personal computer), a laptop, and/or a PC.

The medical staff device 100 may receive prediction results associated with the doppler ultrasound images of the individual from the information providing server 300 and display the received results through a display unit to be described later.

The information providing server 300 may include a general-purpose computer, laptop, and/or data server, or the like, which performs various operations to determine information related to the doppler ultrasound image based on the doppler cardiac ultrasound image and the B-mode ultrasound image provided from the doppler cardiac ultrasound imaging diagnostic device 200, such as an ultrasound diagnostic device. In this case, the information providing server 300 may be, but is not limited to, a device for accessing a web server providing a web page or a mobile web server providing a mobile website.

More specifically, the information providing server 300 may receive the doppler cardiac ultrasound image and the B-mode ultrasound image from the doppler cardiac ultrasound imaging diagnostic device 200, classify an ultrasound mode and a view of the received doppler cardiac ultrasound image, and provide information related to the doppler ultrasound image. In this case, the information providing server 300 may classify an ultrasound view from the doppler cardiac ultrasound image using the classification model.

The information providing server 300 may provide the cross-sectional classification results for the doppler ultrasound image to the medical staff device 100.

Information provided from the information providing server 300 in this way may be provided as a web page through a web browser installed on the medical staff device 100, or may be provided in the form of an application or program. In various embodiments, such data may be provided in a form included in a platform in a client-server environment.

Next, components of the information providing server 300 of the present disclosure will be specifically described with reference to FIGS. 2A and 2B.

First, referring to FIG. 2A, the medical staff device 100 may include a memory interface 110, one or more processors 120, and a peripheral interface 130. Various components within the medical staff device 100 may be connected by one or more communication buses or signal lines.

The memory interface 110 may be connected to the memory 150 and transmit various data to the processor 120. Here, the memory 150 may include at least one type of storage media among flash memory type, hard disk type, multimedia card micro type, card type memory (for example, SD or XD memory, or the like), a RAM, an SRAM, a ROM, an EEPROM, a PROM, a network storage, a cloud, and blockchain data.

In various embodiments, the memory 150 may store at least one of an operating system 151, a communication module 152, a graphical user interface (GUI) module 153, a sensor processing module 154, a telephone module 155, and an application module 156. Specifically, the operating system 151 may include instructions for processing basic system services and instructions for performing hardware operations. The communication module 152 may communicate with at least one of one or more other devices, computers, and servers. The graphical user interface module (GUI) 153 may process a graphical user interface. The sensor processing module 154 may process sensor-related functions (for example, processing voice input received using one or more microphones 192). The telephone module 155 may process telephone-related functions. The application module 156 may perform various functions of the user application, such as electronic messaging, web browsing, media processing, navigation, imaging, and other processing functions. In addition, the medical staff device 100 may store one or more software applications 156-1 and 156-2 (for example, information providing applications) associated with any one type of service in the memory 150.

In various embodiments, the memory 150 may store a digital assistant client module 157 (hereinafter, DA client module), and thus store instructions for performing client-side functions of the digital assistant and various user data 158.

Meanwhile, the DA client module 157 may acquire the user's voice input, text input, touch input, and/or gesture input through various user interfaces (for example, I/O subsystem 140) provided in the medical staff device 100.

Additionally, the DA client module 157 may output data in audiovisual and tactile forms. For example, the DA client module 157 may output data consisting of a combination of at least two or more of voice, sound, notification, text message, menu, graphic, video, animation, and vibration. Additionally, the DA client module 157 may communicate with a digital assistant server (not illustrated) using a communication subsystem 180.

In various embodiments, the DA client module 157 may collect additional information about the surrounding environments of the medical staff device 100 from various sensors, subsystems, and peripheral devices to construct a context associated with the user input. For example, the DA client module 157 may provide context information along with the user input to a digital assistant server to infer the user's intent. Here, the context information that may accompany the user input may include sensor information, such as lighting, ambient noise, ambient temperature, images of the surrounding environments, video, or the like. As another example, the context information may include the physical state (for example, device orientation, device position, device temperature, power level, speed, acceleration, motion pattern, cellular signal intensity, or the like) of the medical staff device 100. As another example, the context information may include information (for example, processes running on the medical staff device 100, installed programs, past and current network activities, background services, error logs, resource usage, or the like) related to the software state of the medical staff device 100.

In various embodiments, the memory 150 may include additional or deleted instructions, and may further include additional configurations other than those illustrated in FIG. 2A of the medical staff device 100, or may exclude some configurations.

The processor 120 may control the overall operation of the medical staff device 100 and execute various commands to implement an interface that provides information related to the doppler ultrasound image by driving an application or program stored in the memory 150.

The processor 120 may correspond to a computational device such as a central processing unit (CPU) or an application processor (AP). In addition, the processor 120 may be implemented in the form of an integrated chip (IC) such as a system on chip (SOC) in which various computational devices such as a neural processing unit (NPU) are integrated.

The peripheral interface 130 may be connected to various sensors, subsystems, and peripheral devices to provide data so that the medical staff device 100 can perform various functions. Here, it may be understood that the function performed by the medical staff device 100 is performed by the processor 120.

The peripheral interface 130 may receive data from a motion sensor 160, an illumination sensor (light sensor) 161, and a proximity sensor 162, through which the medical staff device 100 may perform orientation, light, and proximity detection functions, or the like. For another example, the peripheral interface 130 may receive data from other sensors 163 (positioning system-GPS receiver, temperature sensor, biometric sensor), through which the medical staff device 100 may perform functions related to the other sensors 163.

In various embodiments, the medical staff device 100 may include a camera subsystem 170 connected to the peripheral interface 130 and an optical sensor 171 connected to the camera subsystem 170, which may enable the medical staff device 100 to perform various photographic functions, such as taking photographs and recording video clips.

In various embodiments, the medical staff device 100 may include a communication subsystem 180 coupled to the peripheral interface 130. The communication subsystem 180 may include one or more wired/wireless networks and may include various communication ports, radio frequency transceivers, and optical transceivers.

In various embodiments, the medical staff device 100 includes an audio subsystem 190 coupled to the peripheral interface 130, the audio subsystem 190 including one or more speakers 191 and one or more microphones 192, such that the medical staff device 100 may perform voice-activated functions, such as speech recognition, voice replication, digital recording, and telephony functions.

In various embodiments, the medical staff device 100 may include an I/O subsystem 140 coupled to a peripheral interface 130. For example, the I/O subsystem 140 may control a touch screen 143 included in the medical staff device 100 via a touch screen controller 141. As an example, the touch screen controller 141 may detect a user's contact and movement or cessation of contact and movement using any one of a plurality of touch sensing technologies, such as capacitive, resistive, infrared, surface acoustic wave technology, proximity sensor array, and the like. As another example, the I/O subsystem 140 may control other input/control devices 144 included in the medical staff device 100 via other input controller(s) 142. As an example, the other input controller(s) 142 may control one or more buttons, rocker switches, thumb-wheels, infrared ports, USB ports, and pointer devices such as a stylus.

Next, referring to FIG. 2B, the information providing server 300 may include a communication interface 310, a memory 320, an I/O interface 330, and a processor 340, each component of which may communicate with each other through one or more communication buses or signal lines.

The communication interface 310 may be connected to the medical staff device 100 and the doppler cardiac ultrasound imaging diagnostic device 200 via a wired/wireless communication network to exchange data. For example, the communication interface 310 may receive the doppler cardiac ultrasound image and B-mode ultrasound image from the doppler cardiac ultrasound imaging diagnostic device 200, and transmit information about the determined view to the medical staff device 100.

Meanwhile, the communication interface 310 that enables transmission and reception of such data includes a wired communication port 311 and a wireless circuit 312, and the wired communication port 311 may include one or more wired interfaces, for example, Ethernet, universal serial bus (USB), FireWire, or the like. In addition, the wireless circuit 312 may transmit and receive data with an external device via an RF signal or an optical signal. In addition, the wireless communication may use at least one of a plurality of communication standards, protocols, and technologies, for example, GSM, EDGE, CDMA, TDMA, Bluetooth, Wi-Fi, VoIP, Wi-MAX, or any other suitable communication protocol.

The memory 320 may store various data used in the information providing server 300. For example, the memory 320 may store the doppler cardiac ultrasound image and B-mode ultrasound image, or store a view classification model trained to classify the ultrasound views within the doppler cardiac ultrasound image.

In various embodiments, the memory 320 may include a volatile or nonvolatile storage medium capable of storing various data, commands, and information. For example, the memory 320 may include at least one type of storage media among a flash memory type, a hard disk type, a multimedia card micro type, a card type memory (for example, an SD or XD memory, or the like), a RAM, an SRAM, a ROM, an EEPROM, a PROM, a network storage, a cloud, and blockchain data.

In various embodiments, the memory 320 may store a configuration of at least one of an operating system 321, a communication module 322, a user interface module 323, and one or more applications 324.

The operating system 321 (for example, an embedded operating system such as LINUX, UNIX, MAC OS, WINDOWS, VxWorks, or the like) may include various software components and drivers to control and manage general system operations (for example, memory management, storage device control, power management, or the like) and may support communication between various hardware, firmware, and software components.

The communication module 322 may support communication with other devices through the communication interface 310. The communication module 323 may include various software components for processing data received by the wired communication port 311 or wireless circuit 312 of the communication interface 310.

The user interface module 323 may receive a user's request or input from a keyboard, touch screen, microphone, or the like through an I/O interface 330 and provide a user interface on the display.

The application 324 may include a program or module configured to be executed by one or more processors 330. Here, the application for providing information associated with doppler ultrasound images may be implemented on a server farm.

The I/O interface 330 may connect at least one of an input/output device (not illustrated) of the information providing server 300, such as a display, a keyboard, a touch screen, and a microphone, to the user interface module 323. The I/O interface 330 may receive user input (for example, voice input, keyboard input, touch input, or the like) together with the user interface module 323 and process a command according to the received input.

The processor 340 is connected to the communication interface 310, the memory 320, and the I/O interface 330 to control the overall operation of the information providing server 300, and can perform various commands for providing information through an application or program stored in the memory 320.

The processor 340 may correspond to a computational device such as a central processing unit (CPU) or an application processor (AP). In addition, the processor 340 may be implemented in the form of an integrated chip (IC) such as a system on chip (SoC) in which various computational devices are integrated. Alternatively, the processor 340 may include a module for calculating an artificial neural network model such as a neural processing unit (NPU).

In various embodiments, the processor 340 may be configured to classify and provide views within the doppler cardiac ultrasound image using the classification models.

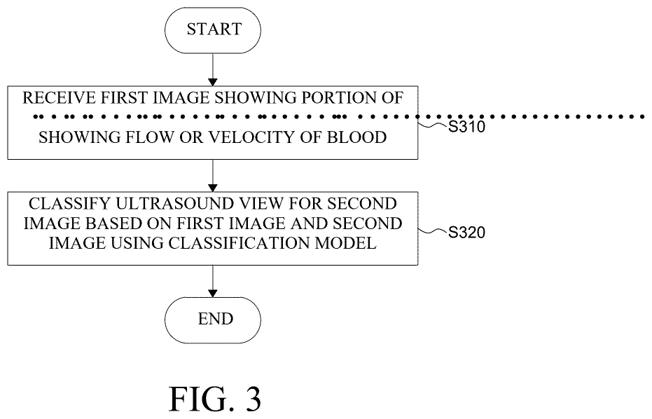

Hereinafter, a method for providing information about a doppler ultrasound image according to one embodiment of the present disclosure will be specifically described with reference to FIGS. 3 to 8.

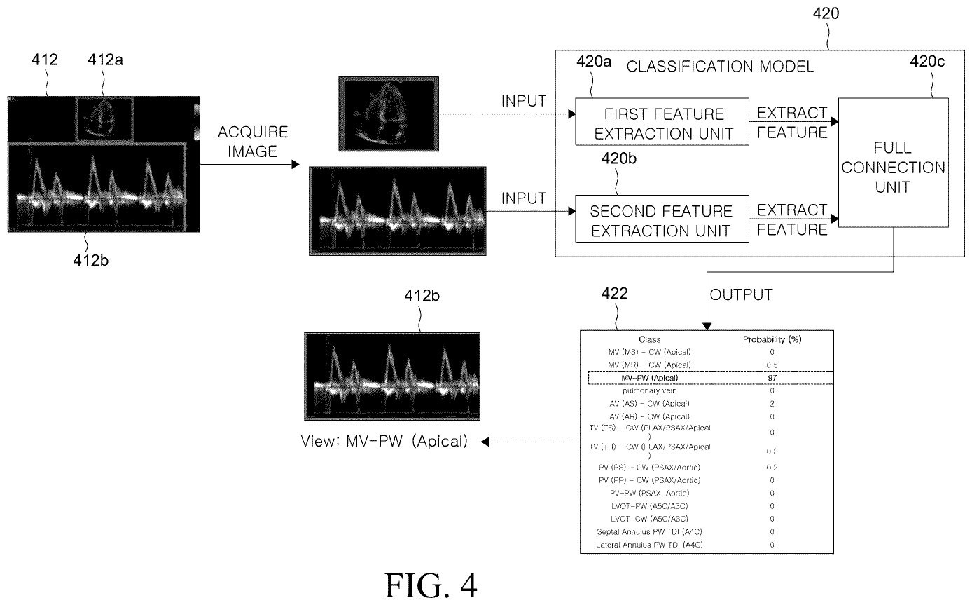

FIG. 3 illustrates a procedure of the method for providing information about a doppler ultrasound image according to one embodiment of the present disclosure. FIGS. 4 to 8 exemplarily illustrate a procedure of the method for providing information about a doppler ultrasound image according to one embodiment of the present disclosure.

First, referring to FIG. 3, the information providing procedure according to one embodiment of the present disclosure is as follows. First, a first image and a second image of an individual are received (S310). Then, the view for the doppler ultrasound image is classified by the view classification model (S320).

More specifically, in a step (S310) where the first image and the second image are received, a doppler cardiac ultrasound image of a target site, that is, a heart site, and a B-mode ultrasound image corresponding to the doppler cardiac ultrasound image may be received.

According to a feature of the present disclosure, in the step (S310) where the first image and the second image are received, a cardiac ultrasound image in DICOM format may be received. In this case, the cardiac ultrasound image in DICOM format may include metadata such as coordinates for the doppler cardiac ultrasound image and the B-mode ultrasound image.

Next, a step (S320) is performed in which the view for the second image is classified.

According to a feature of the present disclosure, the view is a plurality of views, and in the step (S320) where the views for the second image are classified, the probability corresponding to each of the plurality of views for the input doppler image may be calculated and output by the view classification model.

In this case, the classification model may include a first feature extraction unit that extracts features using the B-mode cardiac ultrasound image as input, a second feature extraction unit that extracts features using the doppler cardiac ultrasound image as input, and a full connection unit that integrates features from each of the first feature extraction unit and the second feature extraction unit.

For example, referring to FIG. 4 together, in the step (S320) in which the view for the second image is classified, each of a B-mode cardiac ultrasound image 412a and a doppler cardiac ultrasound image 412b in a cardiac ultrasound image 412 is input to a view classification model 420. In this case, the view classification model 420 includes a first feature extraction unit 420a configured to extract features using the B-mode cardiac ultrasound image as input, a second feature extraction unit 420b configured to extract features using the doppler cardiac ultrasound image as input, and a full connection unit 420c configured to integrate the features extracted from each. Furthermore, the view classification model 420 further includes an output unit (not illustrated) configured to finally output a probability corresponding to each class, that is, a probability corresponding to the view. Here, the output unit (not illustrated) has an output node corresponding to the number of each of the plurality of views, and a probability corresponding to each of the plurality of views for the second image may be output through the output node.

In other words, in the step (S320) where the view for the second image is classified, the features extracted from each of the first feature extraction unit 420a and the second feature extraction unit 420b are integrated through the full connection unit 420c, and finally, a probability 422 for each of the plurality of views calculated for the input doppler cardiac ultrasound image 412b is output through the output unit (not illustrated).

Consequently, based on the probability 422 for each of the plurality of views, the view of the doppler cardiac ultrasound image 412b may be determined as MV-PW (Apical) with the highest probability value of 97%.

Again, referring to FIG. 3, in various embodiments of the present disclosure, prior to the step (S310) of receiving the first image and the second image, a step of receiving an ultrasound image in a digital imaging and communications in medicine (DICOOM) format that indicates metadata including tagging information for a doppler cardiac ultrasound image, and acquiring the B-mode cardiac ultrasound image and the doppler cardiac ultrasound image based on the metadata may be further performed.

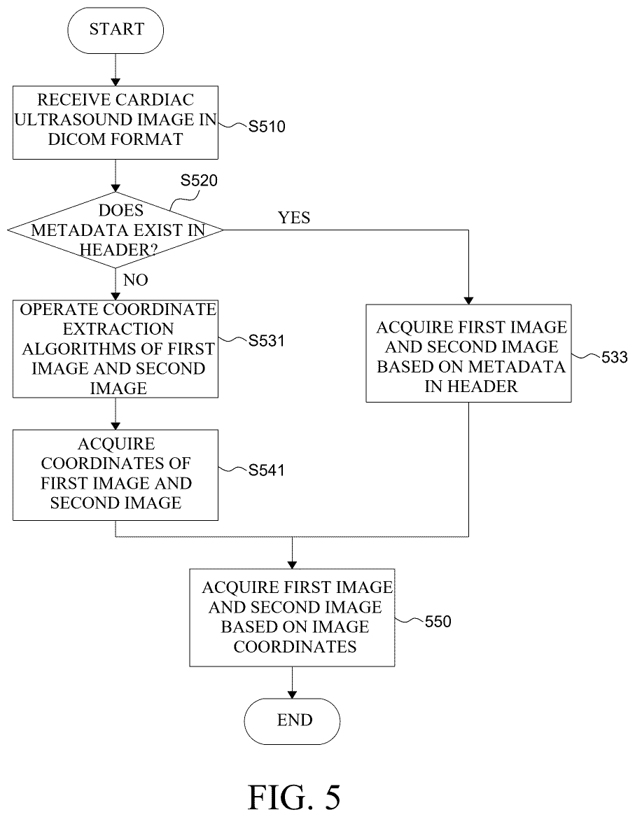

For example, referring to FIG. 5 together, a cardiac ultrasound image in DICOM format is received (S510), and whether metadata exists in a header is determined (S520). At this time, when the metadata does not exist, an algorithm for extracting coordinates of the B-mode cardiac ultrasound image and the doppler cardiac ultrasound image from within the cardiac ultrasound image in DICOM format is operated (S531), and as a result, coordinates of the B-mode cardiac ultrasound image and the doppler cardiac ultrasound image are acquired (S541). Then, the B-mode cardiac ultrasound image and the doppler cardiac ultrasound image may be acquired based on the coordinates (S550). Optionally, when the metadata exists, the coordinates of the B-mode cardiac ultrasound image and the doppler cardiac ultrasound image are acquired based on the metadata in the header (S533), and the B-mode cardiac ultrasound image and the doppler cardiac ultrasound image may be cropped and acquired based on the coordinates (S550).

That is, by the information acquired from the cardiac ultrasound image in DICOM format, it is possible to acquire the doppler cardiac ultrasound image (or region) and the B-mode cardiac ultrasound image (or region) that can be input into the classification model.

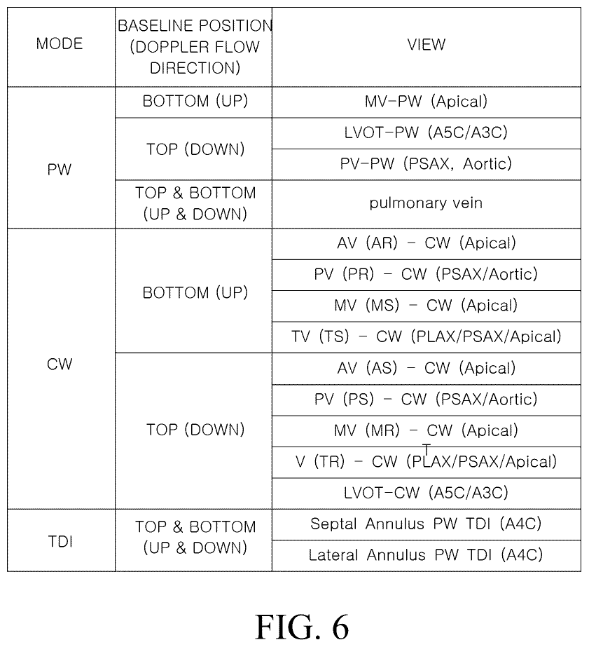

Meanwhile, referring to FIG. 6, the pulsed wave (PW) doppler mode image may have an MV-PW (Apical) view in which the baseline is positioned at the bottom. Furthermore, the pulsed wave doppler mode image may have LVOT-PW (A5C/A3C) and PV-PW (PSAX, Aortic) views in which the baseline is positioned at the top, or a pulmonary vein view in which the baseline exists at the top and bottom.

Furthermore, the continuous wave (CW) doppler mode image may have views of AV (AR)-CW (Apical), PV (PR)-CW (PSAX/Aortic), MV (MS)-CW (Apical), and TV (TS)-CW (PLAX/PSAX/Apical) in which the baseline is positioned at the bottom. Furthermore, the continuous wave doppler mode image may have views of AV (AS)-CW (Apical), PV (PS)-CW (PSAX/Aortic), MV (MR)-CW (Apical), TV (TR)-CW (PLAX/PSAX/Apical), and LVOT-CW (A5C/A3C) in which the baseline is positioned at the top.

Additionally, the tissue doppler imaging mode may have views of Septal Annulus PW TDI (A4C) and Lateral Annulus PW TDI (A4C) in which the baseline is positioned at the top and bottom.

In this way, the view may be classified in detail according to the doppler flow direction, that is, the position of the baseline, in one mode (for example, pulsed wave doppler mode).

Accordingly, referring back to FIG. 3, in the step (S320) where the view for the second image is classified, the view according to the doppler mode of the view for the pulsed wave doppler mode or the view for the continuous wave doppler mode may be classified based on the received first image and the doppler cardiac ultrasound image.

In this case, the doppler cardiac ultrasound image includes the baseline that serves as the reference for the doppler flow direction as described above, and after the step (S320) in which the view for the second image is classified, the view according to the doppler mode may be classified in detail based on the baseline-based algorithm.

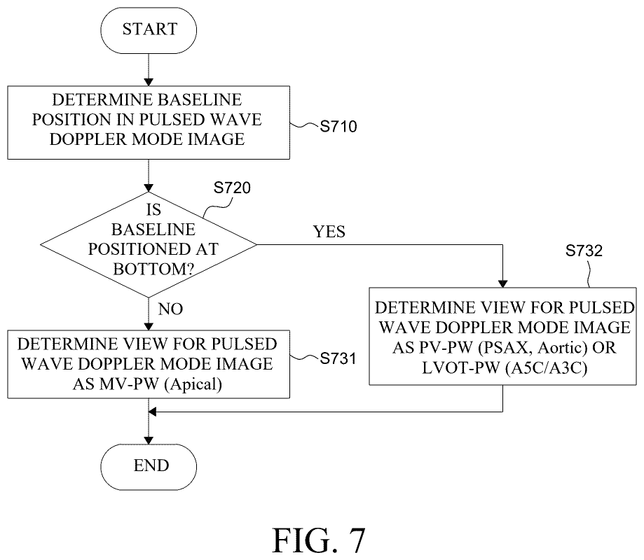

For example, referring to FIGS. 3 and 7 together, as a result of the step (S320) in which the view for the second image is classified, when it is probabilistically determined that the doppler ultrasound image corresponds to a view for the pulsed wave doppler mode, the baseline position in the pulsed wave doppler mode image is determined (S710). Next, it is determined whether the position of the baseline exists at the bottom (S720). In this case, when the position of the baseline exists at the top, that is, when the doppler flow direction is “DOWN”, the view for the pulsed wave doppler mode image may be determined as MV-PW (Apical) (S731). Optionally, when the position of the baseline exists at the bottom, that is, when the doppler flow direction is “UP”, the view for the pulsed wave doppler mode image may be determined as PV-PW (PSAX, Aortic) or LVOT-PW (A5C/A3C) (S732). In this case, based on the probability output from the classification model, the view with the highest value may be determined as the final cross-section.

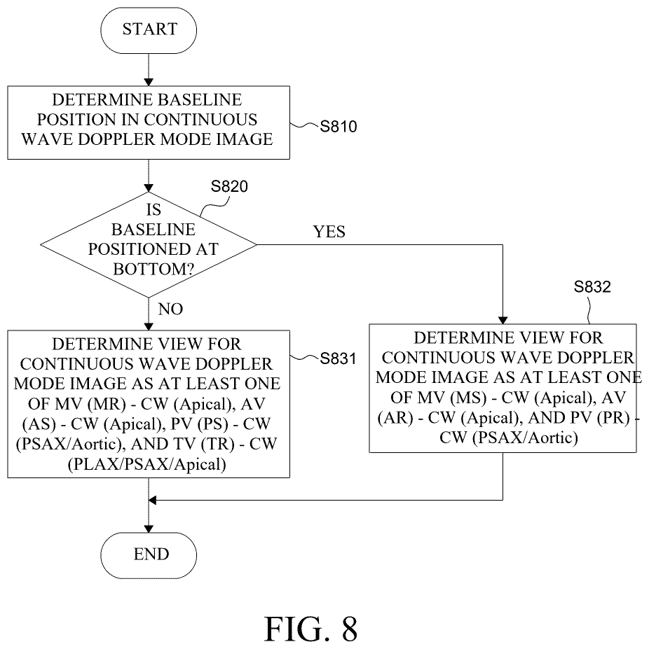

For another example, referring to FIGS. 3 and 8 together, as a result of the step (S320) of classifying the view for the second image, when it is probabilistically determined that the doppler ultrasound image corresponds to the view for the continuous wave doppler mode, the baseline position in the continuous wave doppler mode image is determined (S810). Then, it is determined whether the position of the baseline exists at the bottom (S820). In this case, when the position of the baseline exists at the top, that is, when the doppler flow direction is “DOWN”, the view for the continuous wave doppler mode image may be determined as at least one of MV (MR)-CW (Apical), AV (AS)-CW (Apical), PV (PS)-CW (PSAX/Aortic), and TV (TR)-CW (PLAX/PSAX/Apical) (S831). Optionally, when the baseline position exists at the bottom, that is, when the doppler flow direction is “UP”, the view for the continuous wave doppler mode image may be determined as at least one of MV (MS)-CW (Apical), AV (AR)-CW (Apical), and PV (PR)-CW (PSAX/Aortic) (S832). In this case, based on the probability output from the classification model, the view with the highest value may be determined as the final cross-section.

That is, a postprocessing algorithm based on the doppler flow direction may be applied to the classification results of the classification model, thereby enabling more accurate view classification of the doppler ultrasound images.

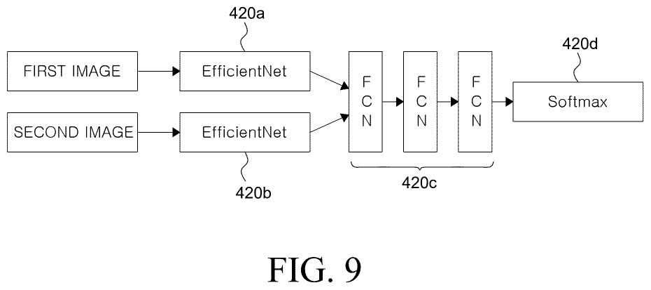

Hereinafter, with reference to FIG. 9, the structural features and learning of an information classification model associated with the doppler ultrasound image used in various embodiments of the present disclosure will be described. In this case, for convenience of explanation, the drawing symbols mentioned above in FIG. 4 are used as references.

FIG. 9 is an exemplary diagram illustrating the structure of the view classification model used in the method for providing information about a doppler ultrasound image according to one embodiment of the present disclosure.

Referring to FIG. 9, the view classification model used in various embodiments of the present disclosure may have a DBNet structure. Specifically, the DBNet-based view classification model 420 may include a plurality of blocks in which various convolution operations are performed.

More specifically, the view classification model includes a first feature extraction unit 420a having an EfficientNET structure configured to extract the features for the input B-mode cardiac ultrasound image (a first image), and a second feature extraction unit 420b having an EfficientNET structure configured to extract the features for an input doppler cardiac ultrasound image (a second image). The features extracted from each of the feature extraction units are integrated through a full connection unit 420c including a plurality of FCNs, and finally, a plurality of classes (views) may be probabilistically classified through an output unit 420d of Softmax.

According to a feature of the present disclosure, the number of nodes of the output unit 420d may correspond to the number of ultrasonic views.

For example, referring back to FIG. 6, an ultrasound view classification model used in various embodiments of the present disclosure may be a model trained to probabilistically classify a total of 15 views: the views corresponding to three pulsed wave (PW) doppler modes, the views corresponding to nine continuous wave (CW) doppler modes, and the views corresponding to two tissue doppler imaging modes.

In this case, the number of output nodes of the output unit 420d of the view classification model may be equal to 15, which is the number of classification target views set in the learning step.

However, the learning data of the view classification model is not limited to that described above, and the structure of the view classification model is not limited to that described above.

Evaluation: Evaluation of the view classification model used in various embodiments of the present disclosure.

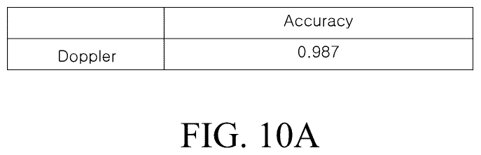

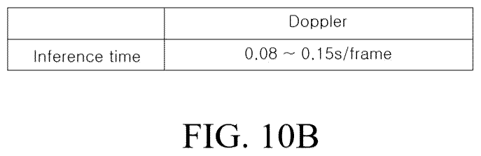

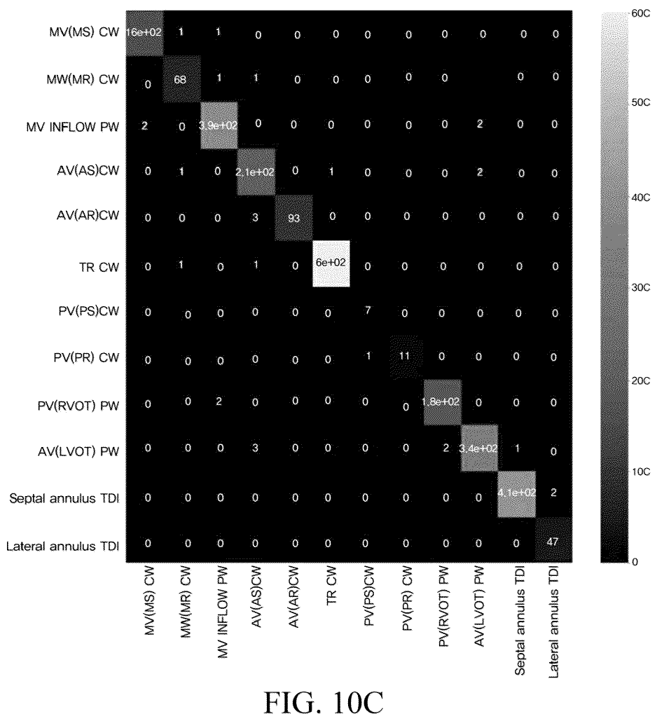

Hereinafter, with reference to FIGS. 10A to 10C, the evaluation results for the view classification model used in various embodiments of the present disclosure are described.

FIGS. 10A to 10C illustrate evaluation results of the view classification model used in the method for providing information according to various embodiments.

First, referring to FIG. 10A, the view classification model used in various embodiments of the present disclosure illustrates a high classification accuracy of 98.7% even though there are 15 cross-sectional classes to be classified.

Next, referring to FIG. 10B, an inference time of the view classification model used in various embodiments of the present disclosure is illustrated to be 0.08 to 0.15 seconds per frame for the doppler ultrasound images. That is, the method for providing information according to various embodiments of the present disclosure can quickly provide information about the view by means of the classification model.

Next, referring to FIG. 10C, doppler confusion matrices for 12 view classification results for the view classification model used in various embodiments of the present disclosure and for the medical staff are illustrated. More specifically, according to the matrix, the view classification model used in various embodiments of the present disclosure illustrates a high degree of confusion in classification for views, which the actual medical staff finds confusing when classifying views.

That is, these results may mean that the view classification model used in various embodiments of the present disclosure has a cross-sectional classification performance at a level similar to that of the actual medical staff.

Although the embodiments of the present disclosure have been described in more detail with reference to the attached drawings, the present disclosure is not necessarily limited to these embodiments, and various modifications may be made without departing from the technical idea of the present disclosure. Accordingly, the embodiments disclosed in the present disclosure are not intended to limit the technical idea of the present disclosure, but to explain it, and the scope of the technical idea of the present disclosure is not limited by these embodiments. Therefore, it should be understood that the embodiments described above are exemplary in all aspects and not restrictive. The protection scope of the present disclosure should be interpreted by the following claims, and all technical ideas within a scope equivalent thereto should be interpreted as being included in the scope of the rights of the present disclosure.

1. A method for providing information about a doppler cardiac ultrasound image implemented by a processor, the method comprising:
receiving a first image showing a portion of an individual's heart and a second image showing a flow or velocity of blood; and
classifying a view for a pulsed wave (PW) doppler mode and a view for a continuous wave (CW) doppler mode based on the received first and second images, by using a view classification model trained to classify the view for a pulsed wave doppler mode and the view for a continuous wave doppler mode using each of the first and second images as input.

2. The method of claim 1, wherein the classification model includes
a first feature extraction unit that extracts features using the first image as input,
a second feature extraction unit that extracts features using the second image as input, and
a full connection unit that integrates the features from each of the first feature extraction unit and the second feature extraction unit.

3. The method of claim 1, wherein
the view is a plurality of views,
the view classification model has an output node corresponding to the number of each of the plurality of views, and
a probability corresponding to each of the plurality of views for the second image is output through the output node.

4. The method of claim 3, wherein the classifying of the view further includes
acquiring the probability corresponding to each of the plurality of views output through the output node, and
determining the view having a highest value among the probabilities corresponding to each of the plurality of views as the view for the second image.

5. (canceled)

6. The method of claim 1, wherein
the second image includes a baseline that serves as a reference for a doppler flow direction, and
the method further includes classifying the view according to the doppler mode in detail based on a position of the baseline after the classifying of the view according to the doppler mode.

7. A method for providing information about a doppler cardiac ultrasound image implemented by a processor, the method comprising:
receiving a first image showing a portion of an individual's heart and a second image showing a flow or velocity of blood, wherein the second image includes a baseline that serves as a reference for a doppler flow direction;
classifying a view for a pulsed wave (PW) doppler mode based on the received first and second images, by using a view classification model trained to classify the view for the pulsed wave doppler mode using each of the first and second images as input; and
determining the view as MV-PW (Apical) when the baseline is positioned at a top of the second image, or determining the view as PV-PW (PSAX, Aortic) or LVOT-PW (A5C/A3C) when the baseline is positioned at a bottom of the second image.

8. The method of claim 6, wherein
the view is a view for the continuous wave doppler mode, and
the classifying in detail further includes
determining the view as at least one of MV (MR)-CW (Apical), AV (AS)-CW (Apical), PV (PS)-CW (PSAX/Aortic), and TV (TR)-CW (PLAX/PSAX/Apical) when the baseline is positioned at a top of the second image, or
determining the view as at least one of MV (MS)-CW (Apical), AV (AR)-CW (Apical), and PV (PR)-CW (PSAX/Aortic) when the baseline is positioned at a bottom of the second image.

9. The method of claim 1, further comprising:
before the receiving,
receiving an ultrasound image in a digital imaging and communications in medicine (DICOM) format that displays metadata including tagging information for the second image; and
acquiring the first image and the second image based on the metadata.

10. The method of claim 1, wherein the classifying of the view further includes, by using the view classification model, determining a doppler mode view of at least one of pulmonary vein, MV (MS)-CW (Apical), MV (MR)-CW (Apical), MV-PW (Apical), AV (AS)-CW (Apical), AV (AR)-CW (Apical), TV (TS)-CW (PLAX/PSAX/Apical), TV (TR)-CW (PLAX/PSAX/Apical), PV (PS)-CW (PSAX/Aortic), PV (PR)-CW (PSAX/Aortic), PV-PW (PSAX, Aortic), LVOT-PW (A5C/A3C), LVOT-CW (A5C/A3C), Septal Annulus PW TDI (A4C), and Lateral Annulus PW TDI (A4C), for the cardiac ultrasound image.

11. (canceled)

12. A device for providing information about a doppler cardiac ultrasound image, the device comprising:
an ultrasound probe configured to provide a first image showing a portion of an individual's heart and a second image showing a flow or velocity of blood; and
a processor functionally connected to the ultrasound probe,
wherein the processor is configured to classify a view for a pulsed wave (PW) doppler mode and a view for a continuous wave (CW) doppler mode based on the received first and second images, by using a view classification model trained to classify the view for a pulsed wave doppler mode and the view for a continuous wave doppler mode using each of the first and second images as input.

13. The device of claim 12, wherein the classification model includes
a first feature extraction unit that extracts features using the first image as input,
a second feature extraction unit that extracts features using the second image as input, and
a full connection unit that integrates the features from each of the first feature extraction unit and the second feature extraction unit.

14. The device of claim 12, wherein
the view is a plurality of views,
the view classification model has an output node corresponding to the number of each of the plurality of views, and
a probability corresponding to each of the plurality of views for the second image is output through the output node.

15. The device of claim 14, wherein the processor is further configured to
acquire the probability corresponding to each of the plurality of views output through the output node, and
determine the view having a highest value among the probabilities corresponding to each of the plurality of views as the view for the second image.

16. (canceled)

17. The device of claim 12, wherein
the second image includes a baseline that serves as a reference for a doppler flow direction, and
the processor is further configured to classify the view according to the doppler mode in detail based on a position of the baseline.

18. The device of claim 17, wherein
the view is the view for the pulsed wave doppler mode, and
the processor is further configured to
determine the view as MV-PW (Apical) when the baseline is positioned at a top of the second image, or
determine the view as PV-PW (PSAX, Aortic) or LVOT-PW (A5C/A3C) when the baseline is positioned at a bottom of the second image.

19. The device of claim 17, wherein
the view is the view for the continuous wave doppler mode, and
the processor is further configured to
determine the view as at least one of MV (MR)-CW (Apical), AV (AS) CW (Apical), PV (PS)-CW (PSAX/Aortic), and TV (TR)-CW (PLAX/PSAX/Apical) when the baseline is positioned at a top of the second image, or
determine the view as at least one of MV (MS)-CW (Apical), AV (AR) CW (Apical), and PV (PR)-CW (PSAX/Aortic) when the baseline is positioned at a bottom of the second image.

20. The device of claim 12, wherein
the ultrasound probe is further configured to provide an ultrasound image in a digital imaging and communications in medicine (DICOM) format that displays metadata including tagging information for the second image, and
the processor is further configured to acquire the first image and the second image based on the metadata.
